(19) United States
(12) Patent Application Publication
(10) Pub. No.: US 2025/0025085 A1
(43) Pub. Date: Jan. 23, 2025

(54) FLEXIBLE BIOSENSOR & ELECTRODE REFINEMENTS

(71) Applicant: COGWEAR, INC., Philadelphia, PA (US)

(72) Inventors: David YONCE, Malvern, PA (US); Benjamin ARCAND, Minneapolis, MN (US); Ryan TETRO, Somerville, MA (US)

(21) Appl. No.: 18/712,651

(22) PCT Filed: Nov. 23, 2022
(86) PCT No.: PCT/US2022/050922

§ 371 Date: May 22, 2024

Related U.S. Application Data
(60) Provisional application No. 63/282,550, filed on Nov. 23, 2021.

(51) Int. Cl. A61B 5/291; A61B 5/00; A61B 5/256; A61B 5/265

(52) U.S. Cl. CPC A61B 5/291; A61B 5/256; A61B 5/265; A61B 5/6803; A61B 2562/0215; A61B 2562/164; A61B 2562/227

(57) ABSTRACT

A flexible, comfortable, and easily repairable EEG signal monitoring device, system and method configured for receiving and analyzing EEG signals and other user and environmental signals that is easily operate and repaired by a user and that is able to correlate received user and environmental data from one or more user to enable users or third part users to make strategic decisions about health, work, police, and military actions.

### PRIORITY

This application claims the benefit of U.S. Provisional Patent Application No. 63/282,550, filed Nov. 23, 2021, the entirety of which is incorporated herein by reference.

### FIELD

The present invention relates generally to electroencephalogram (“EEG”) systems, architectures, and methods related to measuring and monitoring subjects, and, more particularly, to generally flexible biosensors, EEG systems, architectures, electrodes, and methods of use and treatment related thereto.

### BACKGROUND

An electroencephalograph is an electrophysiological monitoring device that is able to record electrical activity of a subject's brain. Since at least the late 1800's scientist have been recording the electrical activities of humans and animals. Electroencephalography (“EEG”) typically includes a number of electrodes that are placed on a subject, typically the head, to record voltage fluctuations or changes that occur from ionic currents within the neurons of the brain.

It was quickly discovered that the voltage fluctuations of the brain had numerous applications. The applications included using the EEG as a diagnostic or clinical tool to diagnose conditions such as epilepsy, sleep disorders, state of consciousness, and even brain death. Even while advancements in medical technology moved forward, such as the invention of the MRI, the EEG's ability to monitor spontaneous changes over time, cements its importance in medicine.

The electrodes of the electroencephalograph conventionally include an adhesive or paste that secures the electrode to the subject's head. Electrodes are also conventionally mounted to or coupled to a holder or substrate such as a headband or head stocking. The electrodes typically include a wire that is coupled to an electroencephalograph that detects the voltage changes and prints the results or findings on a screen or piece of paper that is analyzed by a healthcare worker.

The electroencephalograph generally consists of an electronic circuit including amplifiers and controls for processing the electrical signals received by the electrodes. The electroencephalograph also traditionally included an output device, such as an oscillograph, or more recently, a liquid crystal display, for converting the data into a readable form. All of these devices have traditionally been large, heavy, and generally required to be stationary within a room.

Various attempts have been made to provide EEG systems, architectures, and methods that can be comfortably worn by a subject. While advancements for comfortability for the subject have been made, they have failed to provide EEG systems, architectures, and methods that are needed for modern times.

What is needed and what is provided by the present invention includes having EEG systems, architectures, and methods that are easily mobile and easily used by subjects in vast or remote areas while also providing a clinical-grade signal quality having no to minimal motion artifacts. The present invention also provides EEG systems, architectures, and methods having generally flexible electrodes that are generally conformable to an article of clothing or to a subject's anatomy. Additionally, the present invention is easily replaced, repaired, or exchanged by a subject. Another advantage of the present invention is its ability to operate within a remote network that collects subject data in real-time. Yet another advantage of the present invention is its ability to collect individual subject data while in the subject is in the field and then can transmit, upload, or download the subject's data once the subject is in a secure area or location.

The above is not intended to limit the scope of the invention, or describe each embodiment, aspect, implementation, feature, or advantage of the invention. The detailed technology and preferred embodiments for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

In the accompanying drawings:

FIG. 1 is a perspective view of the fieldable EEG headband in accordance with the embodiments of the invention.

FIG. 2A is a top view an example sensor in accordance with the embodiments of the invention.

FIG. 2B is a cross section view along lines 1-1 of FIG. 2A.

FIG. 3A is a perspective view of a sensor unit being coated in accordance with the embodiments of the invention.

FIG. 3B is a side view showing different layers of a sensor unit in accordance with the embodiments of the invention.

FIG. 4 is a side view of a sensor assembly in accordance with the embodiments of the present invention.

FIG. 5 is a top view of another example sensor unit in accordance with the embodiments of the invention.

FIG. 6A is a top view of an example sensor board in accordance with the embodiments of the invention.

FIG. 6B is a bottom view of the example sensor board of FIG. 6A.

FIG. 7A Fig. is a side view of a sensor assembly in accordance with the embodiments of the invention.

FIG. 7B is a side view of FIG. 7A illustrating a passage opening in the sensor board in phantom lines.

FIG. 8A is a top view of an example sensor board having a plug and play feature in accordance with the embodiments of the invention.

FIG. 8B is a bottom view of the sensor board of FIG. 8A.

FIG. 9 is a side view of multiple sensors arranged on a sensor board in accordance with the embodiments of the present invention.

FIG. 10A is a top view of an example sensor board having a plug and play feature in accordance with the embodiments of the invention.

FIG. 10B is a bottom view of the sensor board of FIG. 10A.

FIG. 10C is a side view of a sensor assembly mounted on the sensor board of FIG. 10B.

FIG. 11A is a top view of a sensor assembly in accordance with the embodiments of the invention.

FIG. 11B is a side view of the sensor assembly of FIG. 11A.

FIG. 12A is perspective view of a sensor assembly in accordance with the embodiments of the invention.

FIG. 12B is side view of a sensor assembly having creases in accordance with the embodiments of the invention.

FIG. 13A is a perspective view of a manufacturing process in accordance with the embodiments of the invention.

FIG. 13B is cross section view along lines 2-2 of FIG. 13A showing silver nano wires deposited in a first layer.

FIG. 13C is a cross section view along lines 2-2 of FIG. 13A showing a conductive PDMS layer deposited on top of the silver nano wire layer.

FIG. 13D is a cross section view along lines 2-2 of FIG. 13A showing a sensor board being placed on the PDMS layer.

FIG. 13E is a perspective view of the final sensor assembly and sensor board.

FIG. 14A is a side view of an example sensor assembly in accordance with the embodiments of the invention.

FIG. 14B is a top view of a circular sensor assembly in accordance with the embodiments of the invention.

FIG. 14C is a top view of a square or rectangular sensor assembly in accordance with the embodiments of the invention.

FIG. 15 is a perspective view of an example sensor assembly in accordance with the embodiments of the invention.

FIG. 16 is a side view of the sensor assembly of FIG. 15 in accordance with the embodiments of the invention.

FIG. 17A is a side view of a sensor unit with a ring member in accordance with the embodiments of the invention.

FIG. 17B is a top view of a circular ring sensor in accordance with the embodiments of the invention.

FIG. 17C is a cross section view along the plane 3-3 of the sensor electrode of FIG. 17A.

FIG. 18A is a top view of a circular ring sensor in accordance with the embodiments of the invention.

FIG. 18B is a top view of a circular ring sensor with a magnet mount in accordance with the embodiments of the invention.

FIG. 19 is a cross section view of a sensor assembly along the lines 4-4 of FIG. 18B.

FIG. 20A is a cross section view of an example sensor assembly in accordance with the embodiments of the invention.

FIG. 20B is a cross section view of an example sensor assembly in accordance with the embodiments of the invention.

FIG. 21A is a top view of a circular ring sensor with openings in accordance with the embodiments of the invention.

FIG. 21A.1 is a detailed view of a portion of FIG. 21A.

FIG. 21B is a cross section view along lines 5-5 of FIG. 21A.

FIG. 22 is a cross section view of an example sensor assembly in accordance with the embodiments of the invention.

FIG. 23 is a cross section view of an example sensor assembly in accordance with the embodiments of the invention.

FIG. 24 is a cross section view of an example sensor assembly in accordance with the embodiments of the invention.

FIG. 25 is a cross section view of an example sensor assembly in accordance with the embodiments of the invention.

FIG. 26 is a cross section view of an example sensor assembly having nano wires encircling the sensor in accordance with the embodiments of the invention.

FIG. 27A is a cross section view of an example sensor assembly having an internal magnet member in accordance with the embodiments of the invention.

FIG. 27B is a cross section view of an example sensor assembly having an internal magnet member and a silver die coating in accordance with the embodiments of the invention.

FIG. 28A is a perspective view of a magnetically attracted member in accordance with the embodiments of the invention.

FIGS. 28B-28D are cross section views along the lines 6-6 of FIG. 28A showing assembly steps of an example sensor in accordance with the embodiments of the invention.

FIG. 29A-29C are cross section views of steps of assembling an example sensor in accordance with the embodiments of the invention.

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the scope of the invention as defined by the appended claims.

### DETAILED DESCRIPTION

The present invention outlines example embodiments of technology and methods related to the manufacture, assembly, detection, interpretation, and response of neural activity in various environments that include, but are not limited to, being away from or remote of a clinical setting. In one example embodiment of the present invention, a user wears an apparatus, such as a headband, that is configured to hold one or more electrodes manufactured according to the method described herein.

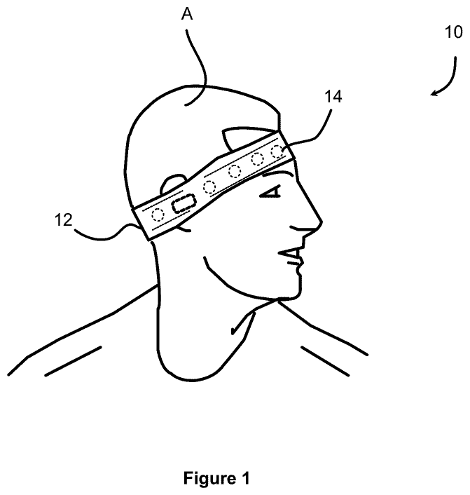

Accordingly, one example embodiment includes flexible electrodes incorporating nanowire conductors within a flexible or generally flexible polymer substrate. The present invention shown, discussed below and illustrated in example FIGS. 1-29C illustrate novel manufacturability, ease-of-assembly, and improved user experience through easier methods to attach and replace electrodes (such as EEG electrodes) on a headband, headgear, or other garment or clothing.

FIG. 1 illustrates the sensor assembly 10 of the present invention incorporated into a headband 12 worn by a user A. The sensor assembly 10 includes one or more sensor units 14 that are manufactured and/or assembled in the various methods and techniques described herein. The sensor assembly 10 also includes one or more power sources that in electrical communication with the one or more sensor units 14 to provide at least selective power thereto. An advantage of the sensor assembly of the present invention is that it combines an exceptional reduction in noise while also reducing the cost to manufacture. Further, it permits user replacement of individual sensors or sensor assemblies.

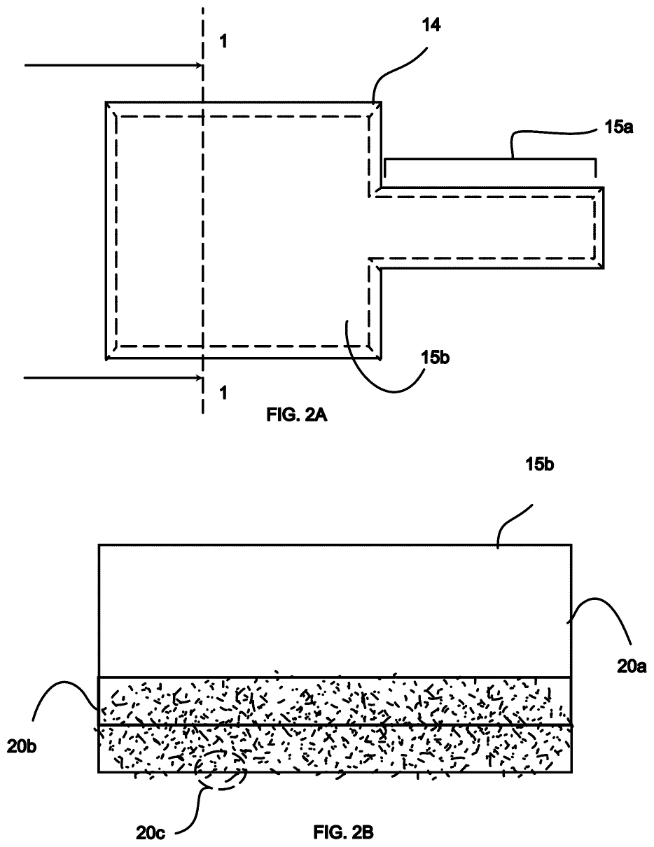

FIG. 2A shows an example embodiment of a basic example outline of a sensor unit 14 of the present invention. While the sensor units 14 will be discussed in relation to EEG sensors, other sensors and sensor technology can also be used. FIG. 2A shows a top of a sensor unit 14 having a tail or connection portion 15a configured to connect to the rest of the sensor assembly 10, and an active portion 15b adjacent to the tail portion 15a and configured to contact a user's skin. In one example embodiment, the active portion 15b of the sensor unit 14 may have a generally larger surface area to ensure optimum contact with the user's skin.

The sensor unit 14 is manufactured in one or more layers that function to optimize the reduction of noise and the ability to easily repair the sensor assembly 10. As illustrated in the example embodiment of FIG. 2B, the sensor unit 14 comprises a PDMS or polymer layer 20a and one or more conductive layers 20b. The conductive layer 20b can comprise nanowires or other conductive material 20d, for example, silver, gold, copper, and aluminum, embedded in to the PDMS layer. As particularly illustrated in FIG. 2B, the conductive material 20c can extend at least partially out of the conductive layer 20b. This can be done to ensure optimum contact with the user's skin.

The conductive layer 20b may extend across a portion or the entire surface area of the active portion 15b. The conductive layer 20b and the conductive material 20c may extend partially or completely through a thickness of the PDMS/polymer layer 20a. The shape of the sensor unit 14 may be any shape, including but not limited to, circular, square, rectangular, triangular, and the like. The conductive material 20c may be manufactured in configurations that define conductive segments or portions that run through a portion of the PDMS/polymer layer 20a.

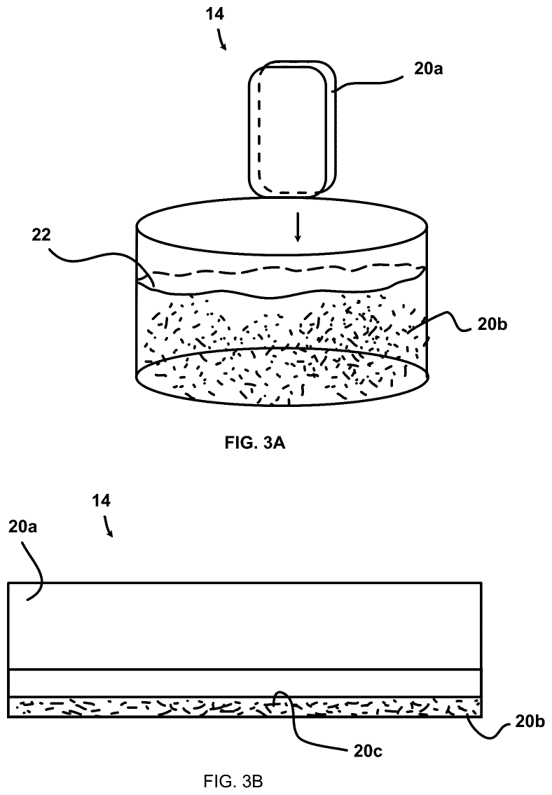

In another example embodiment of the present invention, the conductive material 20c may be placed into an aqueous solution 22 to a concentration needed for the desired conductive properties. As illustrated in FIG. 3A, the PDMS/polymer layer 20a of the sensor unit 14 can be dipped, submerged, or coated with the aqueous solution 22 and then removed and allowed to dry. Once dried, the PDMS/polymer 20a now has conductive layer 20b deposited on it. The dipping or coating process may be repeated several times until a desired thickness of the conductive layer 20b on the PDMS/polymer layer 20a is achieved.

The manner in dipping or coating the PDMS/polymer layer 20a in the conductive aqueous solution 22 depends upon the particular needs. For instance, in one example embodiment, the entire sensor unit 14 may be dipped or coated such that the entire PDMS/polymer layer 20a is coated with one or more layers of the conductive material 20c. In another example embodiment, only a portion of the PDMS/polymer layer 20a is dipped or coated in the aqueous solution 22. For example, FIG. 3B, illustrates a sensor unit 14 having only had a lower portion of the PDMS/polymer layer 20a placed into the aqueous solution 22.

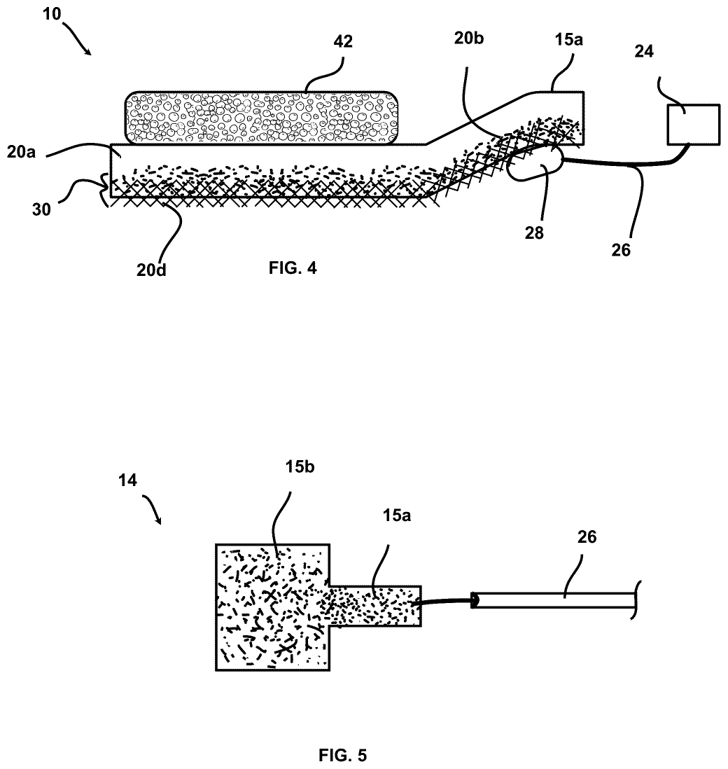

In another example embodiment of the present invention, as illustrated in FIGS. 4 and 5, the conductive material 20c also includes nanowires 20d that be used alone or in combination with other conductive materials 20c. For instance, the nanowires 20d can be coated at least partially in an Ag/AgCl ink coating 30 and then added to or combined with the conductive layer 20b. Alternatively, the nanowires 20d coated with the Ag/AgCl ink coating 30 can be combined with a portion or all of the PDMS/polymer layer 20a. Use of the Ag/AgCl ink coating 30 in conjunction with the Ag allows the sensor unit 14 to stay flexible while overcoming the ionic charge that builds up at the skin-electrode interface.

Once a sensor unit 14 has been made according to one of the above-described methods, it is coupled or connected to the rest of the sensor assembly 10. As illustrated in FIG. 4, the sensor unit 14 can be connected to one or more amplifier circuits 24 via a wire 26 or other conductive member that is bonded or coupled to, or otherwise in direct communication with, a surface of the nanowires 20d or other conductive material 20c. In one example embodiment, the wire 24 or other connecting member 26 is connected to the conductive layer 20b by a conductive bonding agent 28, such as silver epoxy. A conductive bonding agent 28 such as silver epoxy has several advantages over approaches that embed the wire within the PDMS 20a, including providing a simpler and higher-yielding manufacturing process, lower resistance and more reliable connections, and improved strain relief abilities. Additionally, the silver epoxy 28 makes a strong mechanical contact while not damaging the nanowires 20d or other conductive material 20c within the conductive layer 20b. As a result, the sensor unit 14 has increased electrical connectivity and a longer lifetime or longevity.

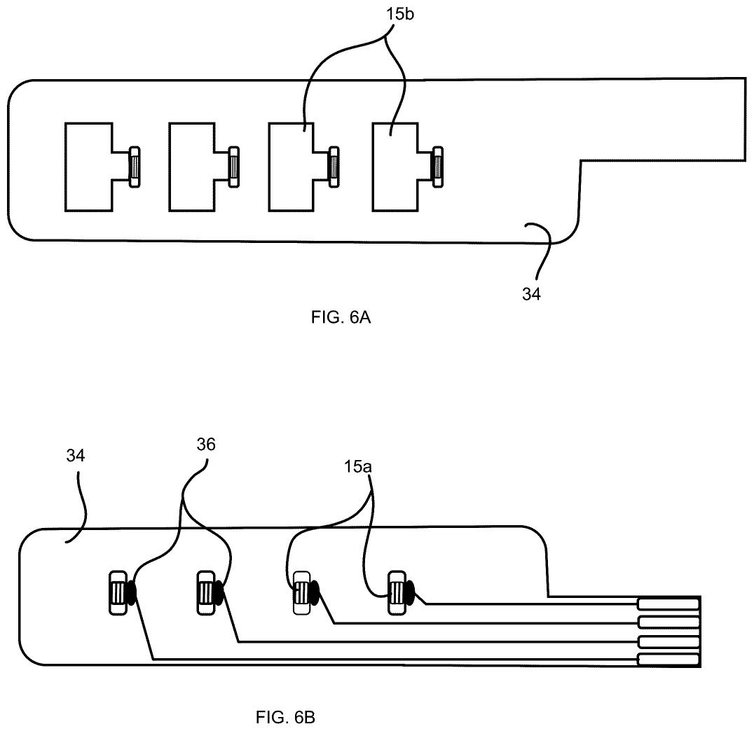

Continuing with FIG. 4, the sensor unit 14 can be manufactured with a curved or “S” shaped tail portion 15a to aid in its assembly with the flexible printed circuit boards 34 of the present invention. As will be discussed in more detail below, the “S” shaped tail portion 15a of the sensor unit 14 is passed to a backside of a board or support member 34 (see FIGS. 6A and 6B), where it can be bonded to a pad or pad region 36 with the conductive bonding agent 28 (e.g., silver epoxy, or another type of bonding member or mechanism). The assembly 10 can be attached to an EEG headgear via a fastening device such as tape, Velcro, or any number of fastening methods. Additionally, the assembly 10 can be attached to the headset or gear for easy removal and replacement.

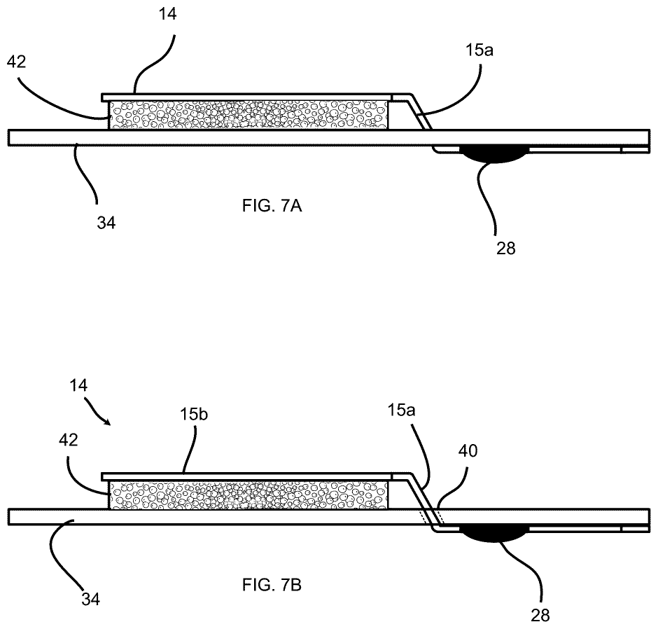

Turning to FIGS. 7A and 7B, the sensor unit 14 with the “S” shaped tail portion 15a is illustrated extending through the flexible sensor circuit board 34. As illustrated in FIG. 7B, the flexible circuit board 34 includes one or more slots or openings 40 formed through it that allows for passage of the tail portion 15a. The one or more slots or openings 40 can have any size and/or shape and may also act as a vent to allow warm air to flow away from a user's skin and cool air to flow towards a user's skins.

The sensor assemblies 10 of the present invention may also comprise one or more cushion or backing members 42 positioned between the active portion 15b of the sensor unit 14 and the flexible printed sensor circuit board 34. The cushion or backing member 42 can comprise any moldable or compressible material (e.g., foam or rubber). The cushion or backing member 42 acts to support the active portion 15b of the sensor unit 14. It also acts as a cushion allowing the sensor unit 14 to move with movement of the user's skin. While the invention is illustrated as having a cushion or backing member 42, it should be considered optional and dependent upon the needs and configuration of the headgear.

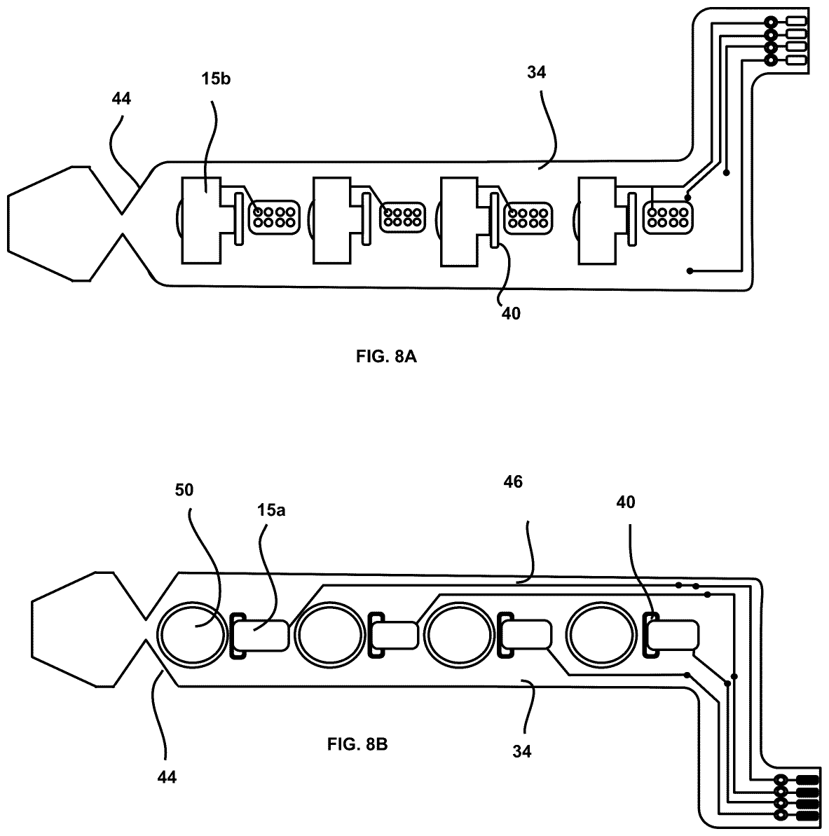

As illustrated in FIGS. 8A and 8B, the flexible printed sensor circuit boards 34 are shown in assembled form with the tail portions 15a of the sensor units 14 extending through the flexible printed sensor circuit boards 34. The flexible printed sensor circuit boards 34 include one or more notches 44 formed therein that are configured to mate with a portion of a user's headgear (helmet, headband, hat, etc.) or another flexible printed sensor circuit board 34. While not shown, other types of fastening or coupling members (e.g., buckles, snaps, clips, etc.) can also be used to secure or removably secure the flexible printed sensor circuit boards 34 to a portion of the user's headgear.

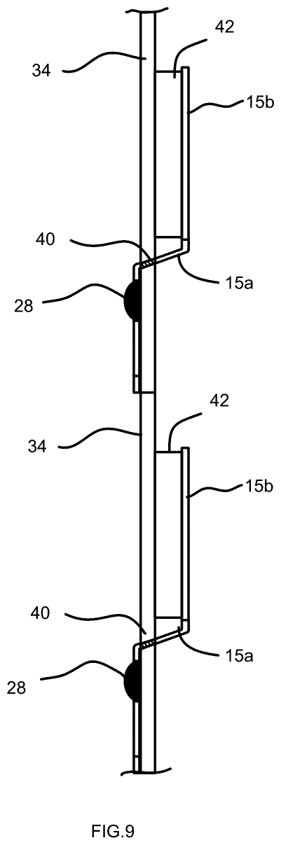

It can be seen in FIGS. 8A-8B and 9 that by having the tail portion 15a of the sensor units 14 extending through to the back or opposite side of the flexible printed sensor circuit boards 34, the width of the flexible printed sensor circuit boards 34 can be reduced since the signal traces 46 are able to be kept together and are not required to go around the larger area of the active portion 15b of the sensor units 14. The narrower flexible printed sensor circuit boards 34 translates into less weight, lower manufacturing costs, and an overall more comfortable unit to be worn by a user.

In another embodiment of the present invention, the sensor assembly 10 includes an attachment/detachment member or assembly 50 that allows for field repair and replacement of the sensor units 14. Referring back to FIG. 8B, attachment/detachment members 50 are shown positioned on the opposite side of the flexible printed senor circuit board 34 (hereafter “PCB 34”) from the sensor units 14. In this particular embodiment, the attachment/detachment members 50 are magnets that are attracted to sensor units or electrodes 14 of the present invention having or containing another magnet or magnetically attracted material that holds the sensor unit or electrode 14 on the PCB 34. The attachment/detachment members 50 also cause the sensor unit 14 to make electrical contact with a pad or pad region 36 on the PCB 34, which can be located on the front surface or back surface of the PCB 34. In FIG. 8A, the pad region can be positioned beneath sensor unit 14 identified as 15b. This method of attachment allows for the simple removal and replacement of electrodes or sensor units 14. Electrodes can also be formed as disclosed in U.S. Provisional Patent Application No. 63/154,751 filed on Feb. 28, 2021.

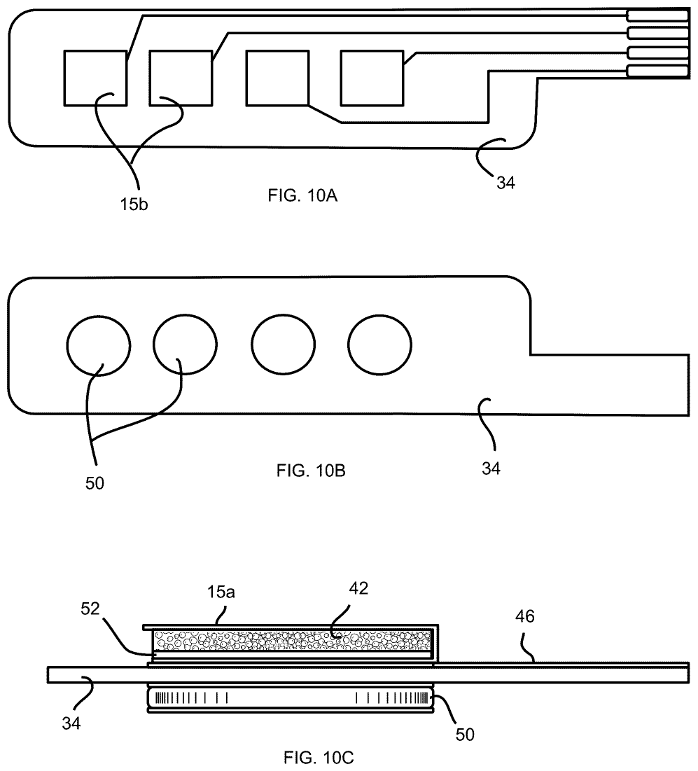

As illustrated in FIGS. 10A and 10B, use of the attachment/detachment members 50 allow each sensor unit or electrode 14 to be discrete on the PCB 34. In another example embodiment of the present invention employing attachment/detachment members 50, a magnetically attracted member 52 (e.g., nickel sheet, magnet, etc.) can be positioned between the sensor units 14 and the PCB 34, with attachment/detachment members 50 positioned on the opposite side of the PCB 34 to hold the sensor unit 14 against the PCB 34. The magnetically attracted member 52 can also be positioned between the cushion member 42 and the PCB 34.

The magnetically attracted member or layer 52 can be attached to a portion of the sensor unit 14 or cushion member 42 by any attaching method (e.g., Velcro, tapes, adhesives, etc.). In another example embodiment, the magnetically attracted member or layer 52 can be embedded in the PDMS layer 20a and/or the cushion member 42 to reduce the overall thickness of the sensor unit 14 on the PCB 34.

The attachment/detachment members 50 can be temporarily or permanently attached to the PCB 34 so that they remain in place when a user pulls off the sensor unit 14 during replacement. This simplifies the process of replacing a sensor unit 14 and allows a user to replace a sensor unit 14 without having to remove the headset or PCB 34. Since the magnetically attracted member or layer 52 remains attached or coupled to the PCB 34 relocating the sensor unit 14 location is simplified as the magnetically attracted member or layer 52 is automatically attracted to attachment/detachment members 50. Further, the sensor unit 14 self-centers or aligns on the PCB 34 eliminating the need for the user to take the time to precisely place the sensor unit 14 on the PCB 34.

Turning to FIGS. 11A and 11B, an example wrapped sensor unit 14 configuration is illustrated showing the conductive layer 20b folded or wrapped around a portion of the PCB 34, or as discussed later, a magnetic attachment member. In this embodiment, the PCB 34 includes electrical contacts or pads 36 on both of its side surfaces. The conductive layer 20b, containing a conductive material, such as the silver nanowires 20d or other conductive materials 20c wraps around from a first or larger planar front side to a second or larger planar back side to make electrical contact between the conductive layer 20b and the contact pads 36 of the PCB 34.

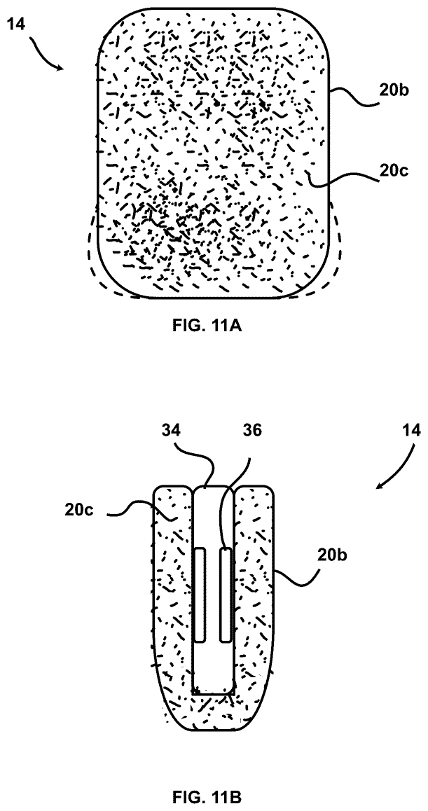

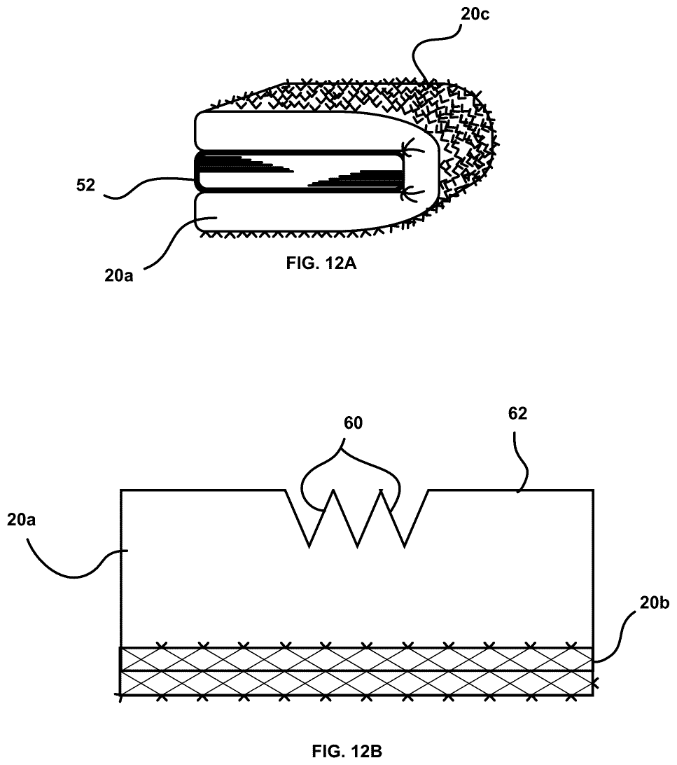

In another example of the wrapped sensor unit 14, as illustrated in FIGS. 12A and 12B, the PDMS layer 20a and/or conductive layer 20b is manufactured with one or more grooves 60 formed on its inner or backside surface 62 to enhance its folding around a tighter radius. The grooves 60 also prevent the sides of the PDMS layer 20a from bulging out at the site of the bend (as seen in the dashed lines of FIG. 11A).

In another example embodiment of the wrapped sensor unit 14, as illustrated in FIG. 12A, the PDMS layer 20a and/or conductive layer 20b (with or without grooves 60) is folded over or around a magnetically attracted member or layer 52. In this embodiment, the conductive layer 20b and/or conductive material 20b is on the outside surface of the sensor unit 14. In this way, when the sensor unit 14 is placed on the PCB 34 the magnetically attracted member 52 in the center of the sensor unit 14 presses the conductive layer against the contacts on the PCB 34.

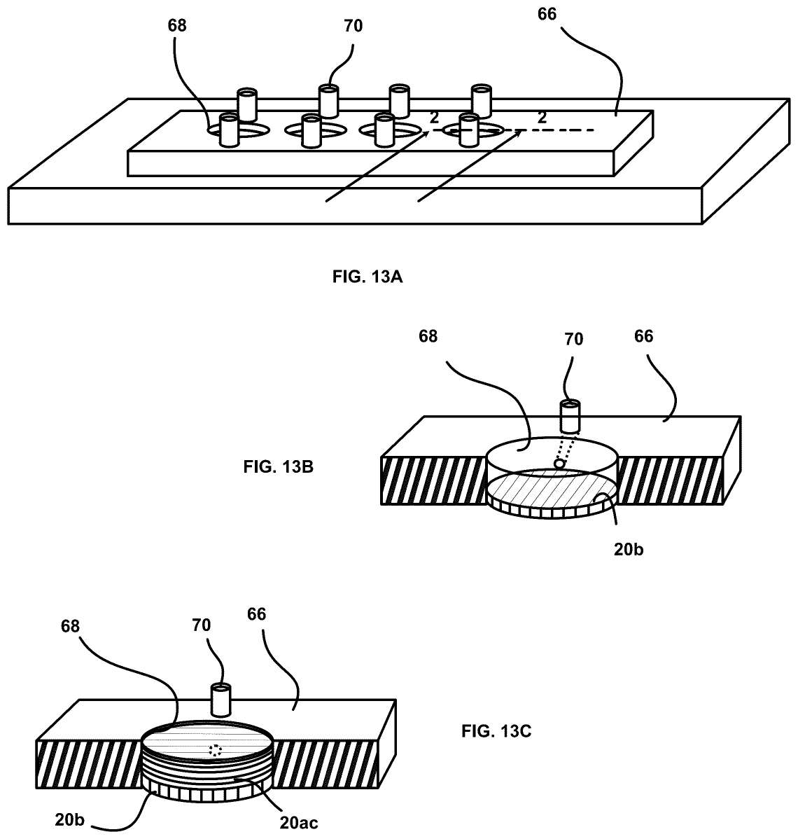

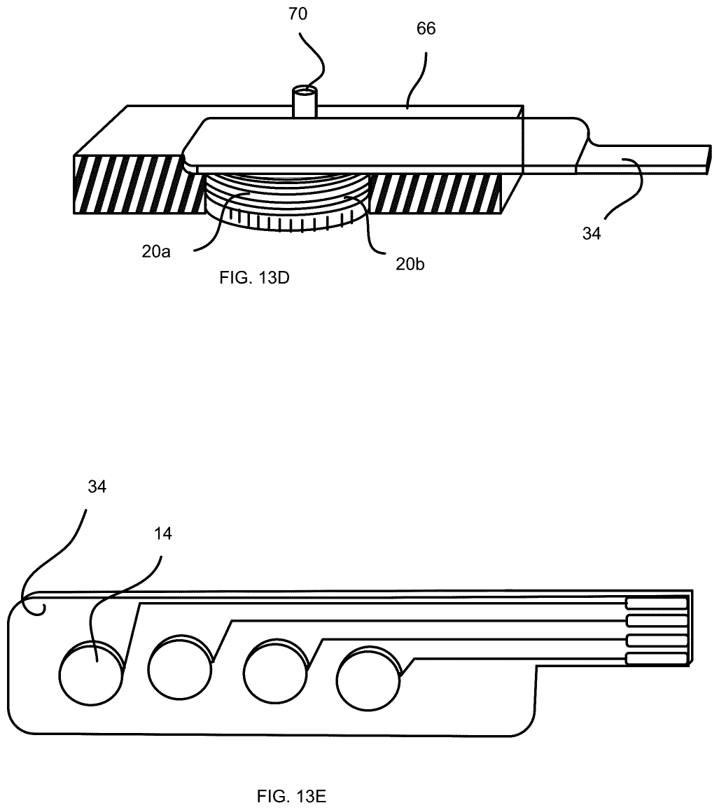

A method of making one or more sensor units 14 and/or sensor assemblies 10 is illustrated in FIGS. 13A-13E. The assembly method illustrates several sensor units 14 being made and connected to a PCB 34. FIG. 13A shows a sensor jig or mold 66, having one or more wells 68 extending into it, positioned on a flat surface such as a table or bench. In the cross section view of FIG. 13B, a conductive layer 20b is deposited in the bottom of the wells 68. Next, as illustrated in FIG. 13C, a layer of PDMS 20a, which may be conductive or not conductive, is layered on top of the conductive layer 20b containing conductive material 20c such as the silver nanowires 20d. The conductive PDMS layer 20a is made by mixing it with a conductor, such as graphene or carbon nanotubes. The conductive PDMS layer 20a creates a conductive path between the surface of the nanowire 20d or conductive layer 20b and the pad 36 on the printed circuit board 34.

The PDMS conductive layer 20a is poured into the wells 68 until filled. With the PDMS conductive layer 20a still wet, a PCB 34 is placed above the sensor mold 66. The pads 36 of the PCB 34 are aligned over each of the wells 68 and then laid on the still-wet PDMS conductive layer 20a until dried. Once dried, the PCB 34 can be lifted from the sensor mold 66, which pulls the now-combined conductive layer 20b and PDMS conductive layer 20a out of the wells 68. A conductive coating, such as Ag/AgCl, may optionally be applied over the cast conductive PDMS layers 20a. The sensor units 14 have now been constructed directly on the PCB 34.

In another example embodiment, a sensor mold 66 can be placed onto the PCB 34 and the PDMS layers 20a cast in a reverse manner. The non-conductive PDMS 20a back layer could be cast first and then another sensor mold 66 with larger mold cavities or wells 68 placed around the cured PDMS pads 36. The peripheral spaces of the larger mold 66 allow for the casting of the conductive PDMS layer 20a+conductive “nanowire” layer 20b to envelope the non-conductive PDMS pad and also make electrical contact with the underlying surface pads 36 of the PCB 34.

Alternatively, the conductive layer of nanowires+PDMS (20b and 20ac, respectively (see FIG. 13C)) can be cast in a sensor mold 66 filled from an edge rather than a large face surface with one of the walls of the mold 66 being (or holding) the PCB 34. This can coat the surface(s) of PCB 34 and skin-facing side of the mold 66 with conductive nanowire+PDMS (20b and 20ac, respectively) making the electrical contact with the PCB pads 36. The “pocket” of conductive nanowires+PDMS (20b and 20ac, respectively) would then be filled with non-conductive PDMS and removed from the mold 66 to create the electrodes.

Yet another embodiment uses a closed mold 66 that is pervious to the nanowire+PDMS solvent allowing an entire inner surface of the mold to be coated with a conductive nanowire+PDMS layer (20b and 20ac, respectively). A port 70 could then be opened to fill in the space with non-conductive PDMS 20a. If less non-conductive PDMS 20a is used than the mold volume, then a hollow electrode 14 can be created that has very compliant properties. The mold 66 can be left static with a preferred orientation to gravity that preferentially thickens the layers (20b and 20a, respectively) on one side of the electrode 14 or the mold 66 can be turned in multiple rotational axes to evenly coat the mold 66 interior (rotomold).

The PCB 34 can have slots and slots of stent-like patterns under and around the PDMS electrodes 14 to allow the pad 36 to flex on the PCB 34 surface more easily. There can also be a large opening under in the PCB 34 and under the PDMS electrodes 14 to allow the electrode surface to flex more easily and also be pushed proud of the surface with an additional feature or material (e.g. a foam pad)

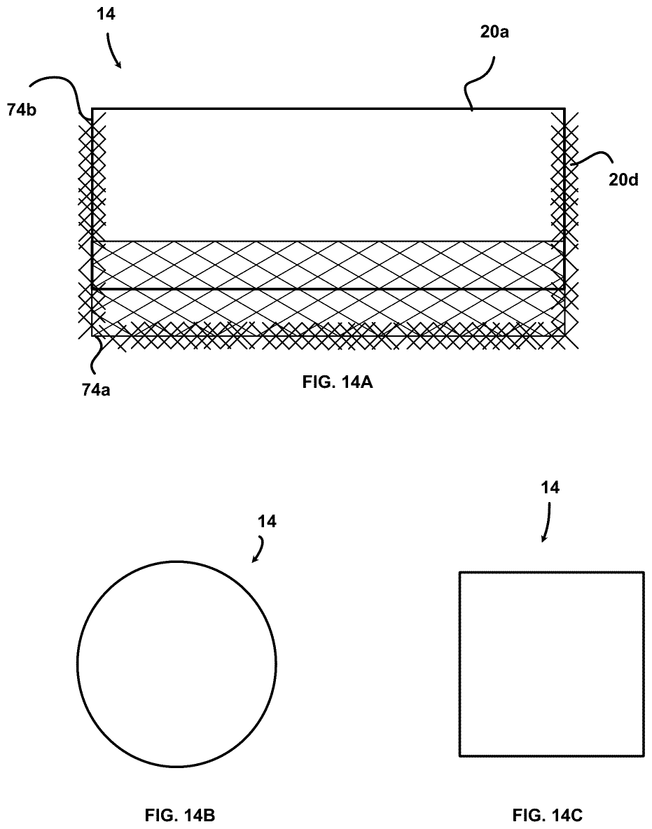

As briefly discussed above, the sensor units 14 can be made a homogeneous conductive or nanowire layer 20b that covers the entire active surfaces (e.g., bottom and vertical portions) of the sensor electrode 14 side surfaces. This embodiment is illustrated in FIGS. 14A-14C. As can be seen in FIG. 14A, the conductive material 20c and or silver nanowires 20d cover the bottom surface 74a and vertical surfaces 74b of the sensor units 14. As illustrated in FIGS. 14B and 14C, the sensor units or electrodes 14 may be made or formed in any shape.

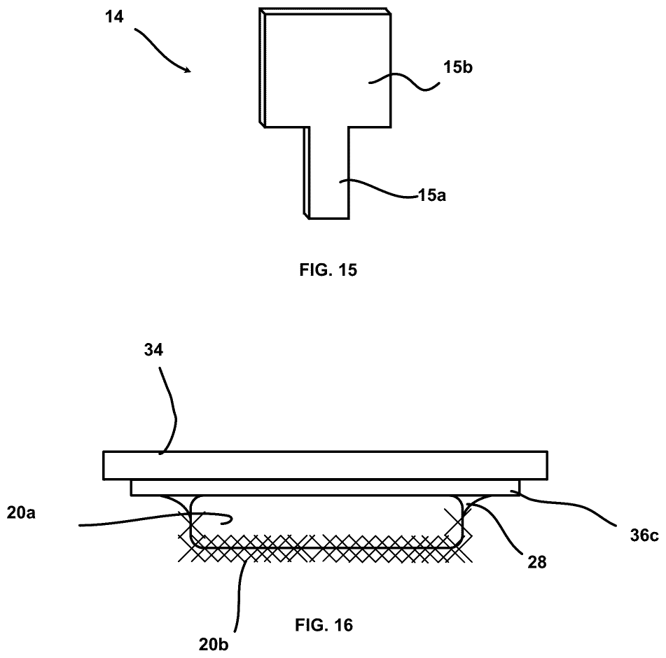

Turning to FIGS. 15 and 16, another sensor unit or electrode 14 configuration of the present invention is illustrated that is able to make a connection directly to a printed circuit board 34 pad 36 at the edges. Alternatively, a bonding agent 26, such as silver epoxy could be used to bond the senor unit or electrode 14 to the PCB 34 and form a reliable and high conductivity connection.

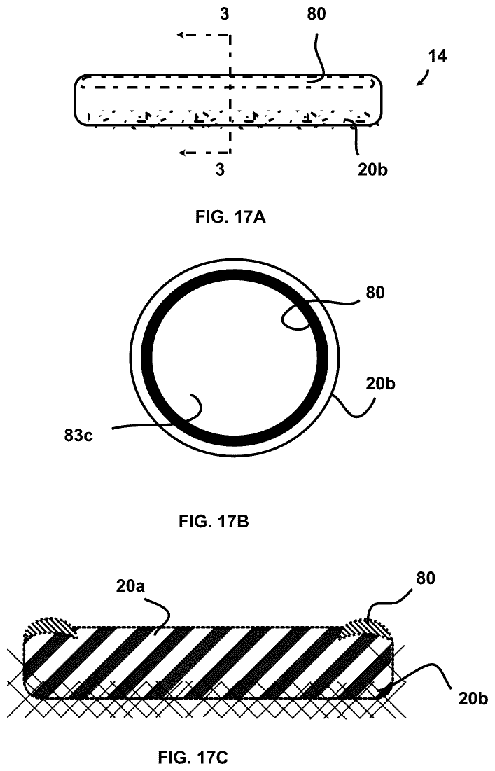

Referring to FIGS. 17A-17C, the sensor unit 14 can be made using a ring member 80 positioned in or connected to the PDMS layer 20a or the conductive layer 20b. The ring member 80 can have a concave lower surface and a convex upper surface, where the convex upper surface is generally flush with or extends beyond an upper surface of the PDMS layer 20a or the conductive layer 20b. As particularly illustrated in FIG. 17B, the ring member 80 can have an open or closed center area 83c.

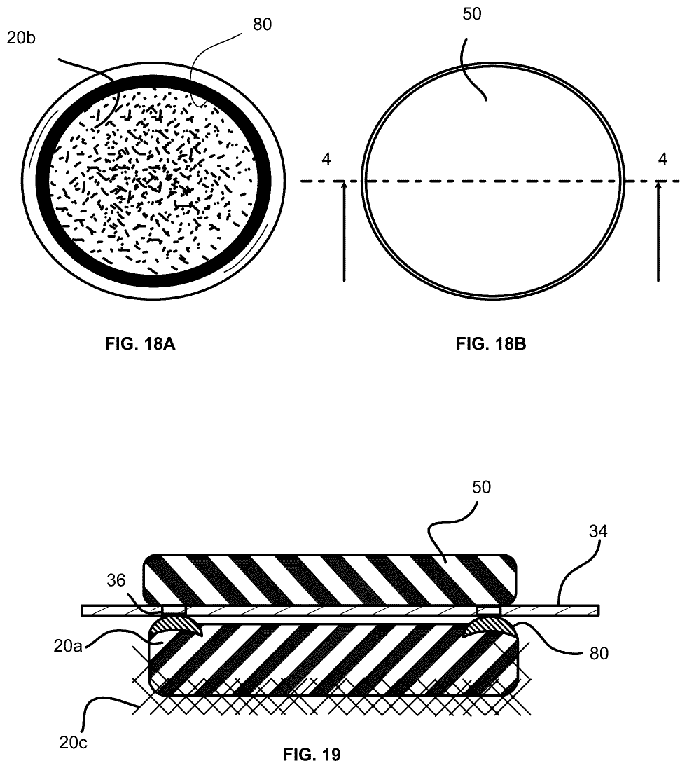

Turning now to FIGS. 18A-18B and 19, the ring member 80 of the sensor unit 14 can be used with the magnet or attachment member 50. The ring member 80 magnetically attracts the attractant member 52 to secure the sensor unit 14 to the PCB 34. The ring member 80 can also act as a conductive member to abut and communicate with the pads 36 on the PCB 34. The pad may be a single solid electrical pad or alternatively multiple pads or shaped to match the dimensions of the ring member 80.

The conductive layer 20b or silver nanowires 20d can extend about and/or through the sensor unit 14 and contact a portion of the ring member 80 to provide a dedicated path from the user's skin to the PCB 34. In other words, the metal ring member 80 bridges the electrical connection from the nanowire surface 20d to the electrical contact pad 36 on the PCB 34. While the embodiments discussed relate to a round sensor unit or electrode 14 and ring member 80, the senor unit or electrode 14 and/or conductive ring member 80 can take any shape and configuration. Additionally, the sensor unit or electrode 14 and/or conductive ring member can have any three dimensional shape and configuration.

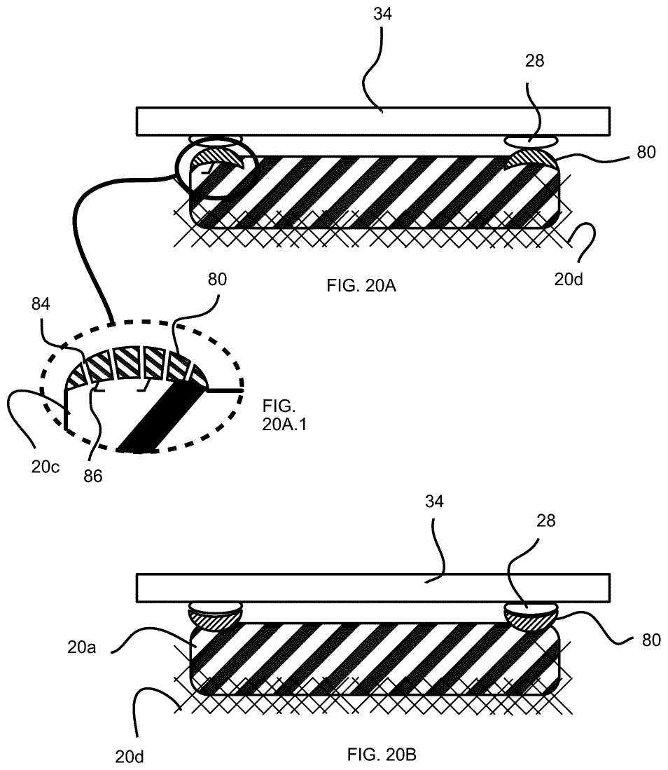

The ring member 80 can alternatively be constructed with holes 84 perforating its bottom surface (see FIG. 20A.1). The holes 84 allows PDMS 20a to penetrate both sides of the ring member 80 to increase pullout strength. The ring member 80 can also have a generally mesh or lattice construction to allow for the passage of PDMS 20a, conductive materials 20b and 20c to extend at least partially through it. The ring member 80 can also have one or more tines 86 (radiating inward, outward or both) that can by bent or curved out of plane to provide adhesion or a gripping force to the PDMS layer 20a as well as to provide electrical contact between the conductive layer 20b and an external wire 26 or electrode 14.

Alternatives to the ring member 80 include helical shape or spirals, coiled spring-like members. The helix shape can be configured as a wide and squat spring with an axis that corresponds to the ring concept. Alternatively, the helix axis can be generally curved to form a ring with a diameter of the helix corresponding to the thickness of the ring member 80 and the helix axis curvature corresponding to the diameter of the ring member 80.

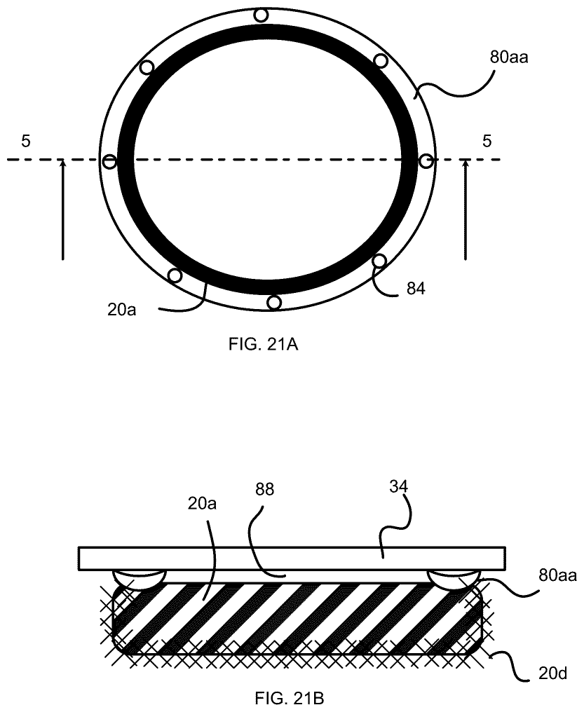

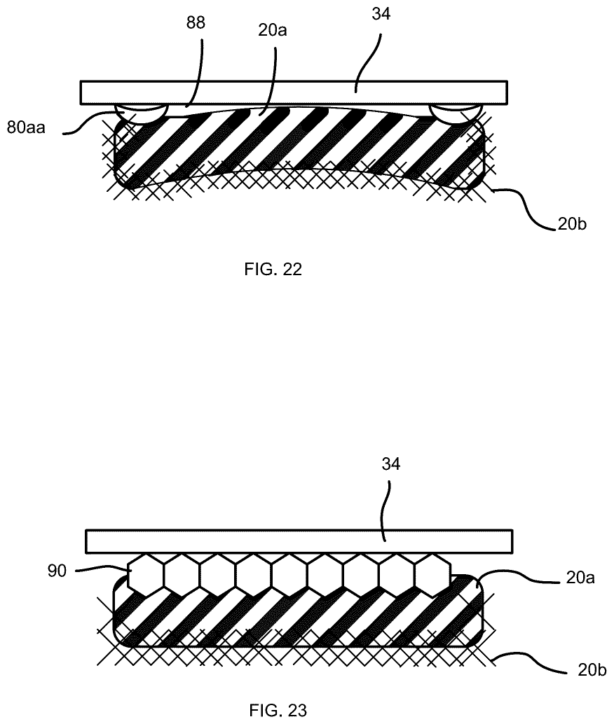

Referring to FIGS. 21A-21B and FIG. 22, the present invention improves comfort and flexibility of the sensor unit or electrode 14, with a modified ring member 80aa that provides a stand-off in a middle or central portion of the sensor unit 14. The stand-off is highly compliant with the user's bodily dimensions, providing more surface area contact and higher comfort than possible with a flatter electrode unit 14 completely bonded on the backside. In this configuration, the sensor unit or electrode 14 is able to flex, bend, or be deformable to accommodate a user's anatomical or clothing features. The deformability of the senor unit or electrode 14 increases comfortability to the user.

A particularly illustrated in FIG. 21B, the modified ring member 80aa is positioned with the concave surface 82a facing the PCB 34 and the convex surface 82b mated with the PDMS layer 20a of the sensor unit or electrode 14. In this configuration the modified ring member 80aa creates a space or void 88 between the PCB 34 and the sensor unit 14. The orientation of the modified ring member 80aa and the space or voids 88 allows the sensor unit or electrode 14 to move and flex with the user.

An example of the type of movement the sensor unit or electrode 14 is capable of in this configuration is shown in FIG. 22. As can be seen, the sensor unit or electrode 14 has flexed or bent into and is capable of occupying, at least temporarily, the space or void 88. Additionally, the modified ring member 80aa is also able to flex lengthwise and/or along its width while still maintaining contact with the PCB 34. As with other embodiments of the invention, the conductive layer 20b or conductive material 20c (e.g., nanowires 20d) are distributed through the sensor unit or electrode 14, whereby they come into contact with the modified ring member 80aa to create a communication pathway for the EEG signal.

The sensor unit or electrode 14 can also, or alternatively, include a 3D mesh structure or contacts 90 that is in contact with the conductive layer 20b or conductive material 20c (e.g., nanowire 20d). The mesh contacts 90 can be embedded in the polymer layer 20a with its top surface exposed to contact a PCB 34 or another surface. The 3D mesh contacts 90 can be constructed from a conductive fabric, such as a silver impregnated or silver woven fabric having various knit patterns. The 3D mesh contacts 90 can be coupled or connected by any means disclosed herein, including but not limited to the wires of the tail portion 15a, adhesives, silicones, and the like. The coupling method or means can also act as an insulator to insulate the connection. The 3D mesh contacts or structure 90 is also able to push through and connect to a portion of the PCB 34. In another example embodiment, the coupling means also comprises an electrically conductive material such as the silver epoxy bonding agent 28 discussed earlier.

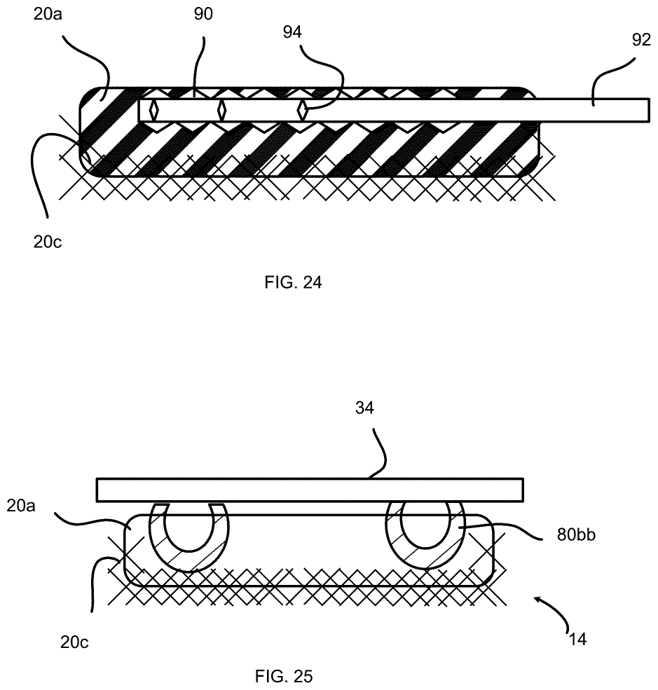

As illustrated in FIG. 24, the mesh contacts or fabric 90 can also extend beyond the polymer/Ag nanowire layer 20c for connection to a circuit via a flexible fabric tail 92 or inserted into a crimp connector 94 disposed in the PDMS layer 20a.

A taller ring member 80bb can be used and be embedded into the PDMS layer 20a to make contact directly with the conductive layer 20b and the conductive material 20c contained therein (e.g., silver nanowire). As can be seen in FIG. 25, the tall ring member 80bb can extend through the backside of the sensor unit or electrode 14. In this manner, the tall ring member 80bb carries the current from the conductive layer 20b and silver nanowires 20d to a PCB 34. It can also be attached to the PCB 34 through a magnetic attachment member 50 or silver epoxy bonding agent 28, as discussed above.

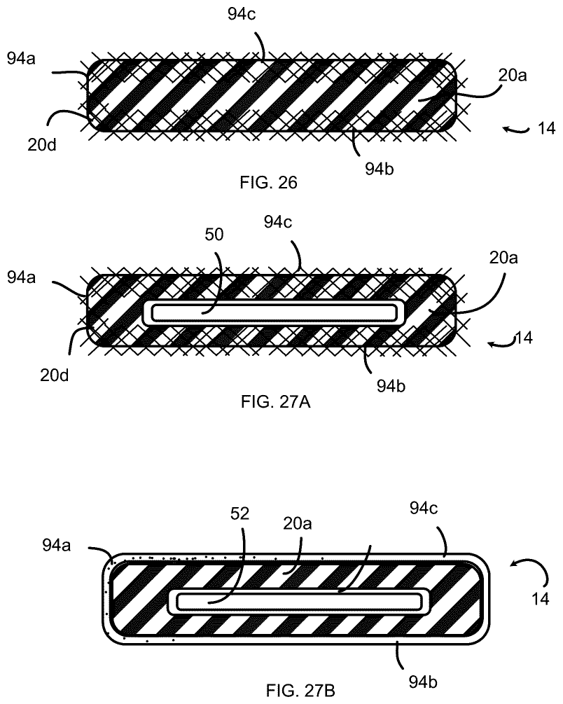

While the conductive layer 20b has been described as extending at least partially around the sensor unit or electrode 14, it is also contemplated herein that the conductive layer 20b can extend completely around an entire outer surface of the sensor unit or electrode 14. As illustrated in FIG. 26, the conductive material 20c or silver nanowires 20d fully encompass the active surface, all vertical sides 94a, front side 94b, and at least a portion of the backside 94c. The EEG signal or current is carried from any of the active surfaces 94a and 94b to the backside 94c where it connects and communicates with a portion of the PCB 34.

The encapsulated sensor unit or electrode 14 can be manufactured with a one-sided or two-sided mold with a PDMS layer 20a fill after nanowire 20d deposition (similar to the process discussed above).

As illustrated in FIGS. 27A and 27B, the encapsulated sensor unit or electrode 14 can have an attachment member or magnet 50 or a magnetically attracted member 52 embedded inside it for magnetic attachment.

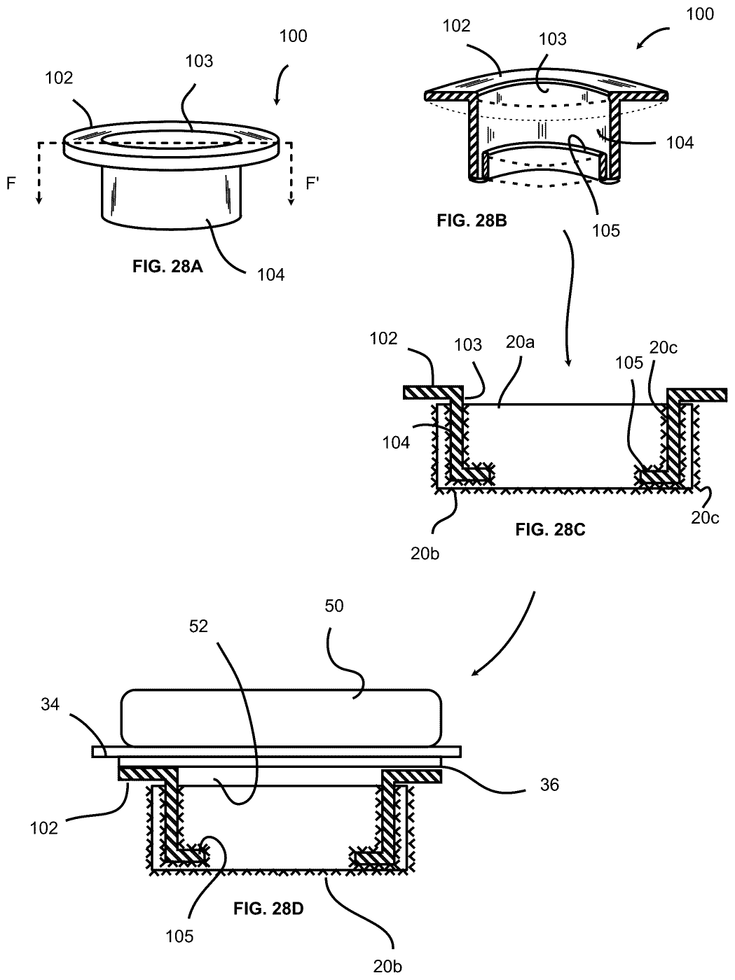

As illustrated in FIGS. 28A-28D, the sensor unit or electrode 14 can include a conductive anchor member 100 having a configuration that helps to anchor it into the polymer layer 20a and/or conductive layer 20b. As illustrated in FIGS. 28A and 28B, the anchor member 100 can have generally J-shape cross section with an outer annular lip 102 defining an opening 103. The annular lip 102 can be exposed outside of the polymer layer 20a when assembled. The outer lip 102 is connected to a J-shaped hook portion or peripheral wall 104 having a curved lip 105 that helps grab onto the polymer layer 20a and/or conductive layer 20b for mechanical stability. The curved lip 105 can be curved inward toward the opening 103 or central axis or outwardly.

The conductive or nanowire layer 20a can encompass both the curved lip 105 of the J-hook peripheral wall 104 and the bottom and sides of the sensor electrodes 14, thereby creating a larger surface area for the conductive layer 20b or nanowires 20d to make electrical contact with the anchor member 100. As with some of the other embodiments, the anchor member 100 bridges the electrical connection from the conductive layer 20b or nanowire surface 20d to the electrical contact pad 36 on the PCB 34. The electrode 14 can be connected to the PCB 34 through magnetic attraction.

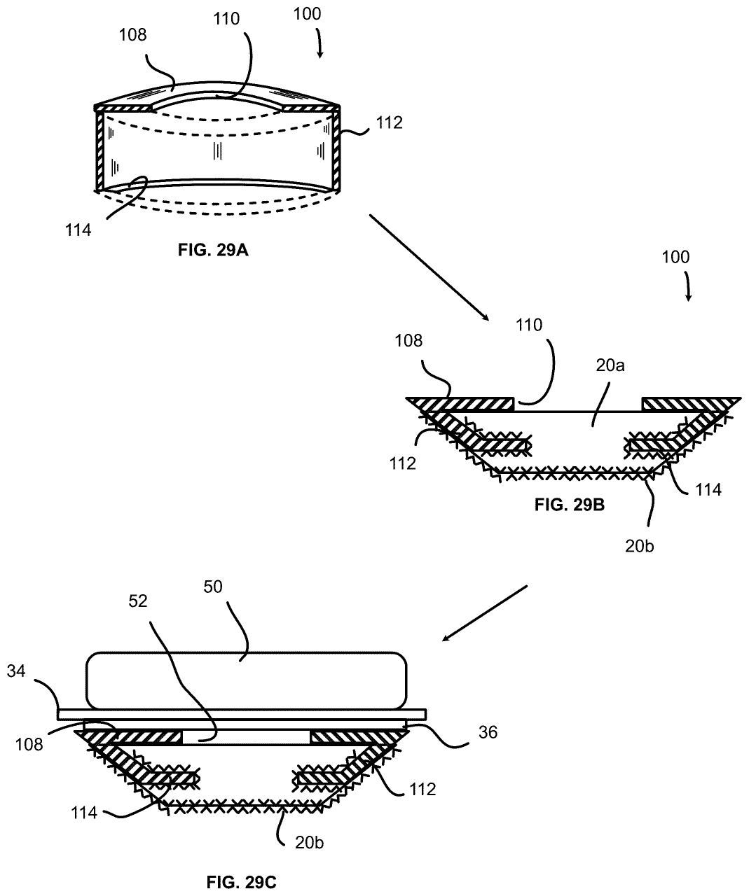

As illustrated in FIGS. 29A-29C, the anchor member 100 can have a generally a cone shape with a conductive inwardly extending ring portion 108 defining an opening 110. Again, the ring portion 108 is generally positioned above the polymer layer 20a when the sensor unit or electrode 14 is assembled. The ring portion 108 is connected to an outer peripheral wall 112 having an inner lip 114 that is generally parallel to the ring portion. The lip 114 can be hooked into the polymer layer 20a and/or conductive layer 20b to help grab onto the layers 20a and 20b for mechanical stability. Additionally, the cone shape of the anchor member 100 aids in anchoring it in the layer 20a and 30b.

The conductive layer 20b or nanowire members 20d can encompass both the inner lip 114 and the bottom and sides of the sensor electrodes 14, creating a larger surface area for the nanowires 20d to make electrical contact with the metal ring portion 108. The metal ring portion bridges the electrical connection from the nanowire 20d surface to the electrical contact pad 36 on the PCB 34. The electrode 14 can be connected to the PCB 34 through magnetic attraction and can be coated with the Ag/AgCl coating.

While the ring is described as “J-Shaped” or “Cone-Shaped” it is understood that any shape may be used. Including but not limited to, U-Shaped, V-Shaped, T-Shaped, and the like.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products. Moreover, features or aspects of various example embodiments may be mixed and matched (even if such combination is not explicitly described herein) without departing from the scope of the invention.

What is claimed is:

1. A repairable sensor headset, comprising:
a support having a first surface and a second opposed surface;
a first attachment member attached to the support;
a signal communication device connected to a portion of the support and configured to transmit a signal from the flexible support;
a sensor assembly removably connectable to the first surface of the support, the sensor assembly comprising:
a conductive substrate having a conductive material component capable of acting as a signal pathway through a portion of the conductive substrate;
a second attachment member coupled to the substrate; and
wherein the first and second attachment members are detachably connectable to removably position the sensor assembly against the support.

2. The repairable sensor headset of claim 1, wherein the conductive material comprises silver nanowires or a silver-silver chloride coating.

3. The repairable sensor headset of claim 1, wherein the first attachment member and the second attachment member are magnetically attracted materials.

4. The repairable sensor headset of claim 3, wherein the first attachment member is positioned on the second opposed surface of the support.

5. The repairable sensor headset of claim 4, wherein the second attachment member is encased in the substrate.

6. The repairable sensor headset of claim 4, wherein the second attachment member is attached to a surface of the substrate positionable proximate to the support.

7. The repairable sensor headset of claim 4, wherein the second attachment member is at least partially enclosed by the conductive substrate.

8. The repairable sensor headset of claim 4, wherein the conductive substrate is folded over the second attachment member forming a first conductive substrate portion connectable to the flexible support and a second conductive substrate portion contactable to a user's head.

9. The repairable sensor headset of claim 1, wherein the conductive material extends across all surfaces of the substrate and configured to contact a portion of the support to for a signal pathway bridge to the support.

10. A repairable sensor system for monitoring a biosignal of a user, comprising:
a flexible sensor support having a first surface and a second surface and at least one conductive pad region disposed on a portion of the first surface or second surface;
a magnetic member attached to the second surface of the flexible sensor support;
a signal communication device connected to a portion of the flexible support and the at least one pad region to transmit a biosignal from the flexible support;
a magnetic sensor assembly connectable to the first surface of the flexible sensor support, the sensor magnetic sensor assembly comprising:
a polymer substrate having a conductive silver chloride coating capable of acting as a signal pathway to the pad region;
an attachment member having magnetic properties coupled to a portion of the polymer substrate; and
wherein the attachment member and magnetic member magnetically secure the magnetic sensor assembly to the first surface of the flexible sensor support where the conductive silver chloride coating is in contact with the conductive pad region where a biosignal is capable of traveling from a user through the along the polymer substrate and into the pad region where it can be transmitted by the signal communication device.

11. The repairable sensor system of claim 10, further comprising conductive nanowires mixed with the polymer substrate to act as an internal biosignal pathway.

12. The repairable sensor system of claim 10, wherein the attachment member is encased in the polymer substrate.

13. The repairable sensor system of claim 10, wherein the attachment member is attached to a surface of the polymer substrate positionable proximate to the flexible sensor support.

14. The repairable sensor system of claim 10, wherein the attachment member is at least partially enclosed by the polymer substrate.

15. The repairable sensor system of claim 10, wherein the polymer substrate is folded over the attachment member forming a first polymer substrate portion contactable to the flexible sensor support and a second polymer substrate portion contactable to a user's head.

16. The repairable sensor system of claim 10, wherein all surfaces of the polymer substrate are coated with the conductive coating and configured to contact a portion of the pad regions for a signal pathway bridge to the flexible support.

17. A method of replacing a biosignal sensor on a flexible sensor support having a magnetic member mounted on at least one of its surfaces comprising the steps:
pulling a magnetic sensor assembly magnetically connected to the surface of the flexible sensor support, the magnetic sensor assembly comprising:
a polymer substrate having a conductive silver chloride coating capable of acting as a signal pathway to a conductive region on the flexible sensor support;
an attachment member having magnetic properties mated with a portion of the polymer substrate;
placing a new magnetic sensor assembly having an attachment member proximate to a location of a removed magnetic sensor assembly on the flexible sensor support;
feeling a magnetic attractive force between the magnetic member and the attachment;
allowing the magnetic attractive force to align the magnetic sensor assembly on the flexible sensor substrate;
releasing the magnetic sensor assembly that is now magnetically connected to the flexible sensor support; and
wherein the silver-silver chloride coating is in contact with the conductive pad region where a biosignal is capable of traveling from a user along the polymer substrate and into the pad region where it can be transmitted by the signal communication device.

18. The method of replacing a biosignal sensor on a flexible sensor support of claim 17, wherein the attachment member is embedded in the polymer substrate.

19. The method of replacing a biosignal sensor on a flexible sensor support of claim 17, wherein the attachment member is attached to a surface of the polymer substrate positionable proximate to the flexible sensor support.

20. The method of replacing a biosignal sensor on a flexible sensor support of claim 17, wherein the attachment member is at least partially enclosed by the polymer substrate.
